(19) United States
(12) Patent Application Publication
(10) Pub. No.: US 2008/0269347 A1
(43) Pub. Date: Oct. 30, 2008

(54) EPINEPHRINE FORMULATIONS

(75) Inventors: Jon Bruss, Cincinnati, OH (US); John McCarty, Miami Springs, FL (US); Blair West, Miramar, FL (US)

Correspondence Address: FULBRIGHT & JAWORSKI, LLP, 1301 MCKINNEY, SUITE 5100, HOUSTON, TX, 77010-3095, US

(73) Assignee: Azopharma, Inc.

(21) Appl. No.: 11/864,234

(22) Filed: Sep. 28, 2007

Related U.S. Application Data
(60) Provisional application No. 60/847,823, filed on Sep. 28, 2006.

(51) Int. Cl. A61K 31/135; A61P 37/08

(52) U.S. Cl. 514/653

(57) ABSTRACT

The present invention generally concerns an epinephrine formulation that has enhanced stability. In particular embodiments, the formulation is an injectable formulation. In specific aspects, the formulation comprises epinephrine, EDTA, and one or more of an antioxidant such as cysteine, citric acid, acetylcysteine, or thioglycerol. The formulations are suitable for any medical condition that is in need of epinephrine, although in specific embodiments the medical condition is anaphylaxis, asthma, or cardiac arrest.

This application claims priority to U.S. Provisional Patent Application Ser. No. 60/847,823, filed Sep. 28, 2006, which is incorporated by reference herein in its entirety.

### FIELD OF THE INVENTION

The fields of the present invention include at least molecular biology, cell biology, and medicine. In certain fields of the invention, there are new compositions of epinephrine and devices and methods of using such epinephrine formulations.

### BACKGROUND OF THE INVENTION

Epinephrine, or (−)-3,4-Dihydroxy-[(methylamino)methyl]benzyl alcohol, is an endogenous adrenergic neurotransmitter synthesized and stored in the adrenal medulla. It is a polar compound characterized structurally by a catechol (a dihydroxybenzene) and an amine, and it is commonly available in a salt form. Epinephrine is water soluble and interacts in a variety of ways, depending on the type of receptor areas of target cells.

Epinephrine is one of the neural hormones responsible for the regulation of the heart, blood pressure, airway resistance, and energy metabolism. It is classified as a sympathomimetic drug, acting on both alpha and beta receptors. Epinephrine generates an inotropic effect, wherein it increases the heart rate, the force of contraction of the heart, narrows the blood vessels thus increasing blood pressure, reduces airway resistance to make it easier to breath, and raises blood glucose and blood fatty acids to supply the body energy during stress. Epinephrine is available in a variety of formulations suited for different clinical indications and routes of administration, for example by injection, by inhalation, or topically. Its uses include at least the following: combating low blood pressure during hemorrhagic or allergic shock; opening the airways during asthmatic attack; restricting the distribution of locally administered drugs such as local anesthetics; reducing nasal congestion; and/or performance aid in emergency situations.

Epinephrine can be prepared synthetically by one of several processes readily available to one in the art. One such process starts with 1,2-dihydroxybenzene that is converted successively to (chloroacetyl)catechol with chloroacetyl chloride, then to (methyl-aminoacetyl)catechol with methylamine and to racemic epinephrine by hydrogenation. The racemic form is resolved with D-tartaric acid to provide a white to nearly-white powder that is sensitive to light, air, heat, or alkaline conditions. Salts with acids are readily formed and provide some stability. The hydrochloride, sulphate, and bitartrate salts are known in the art.

Allergic emergencies, such as anaphylaxis, are a growing concern, given the increasing awareness of members of the public of their frequency and potential severity. Anaphylaxis is a sudden, severe, systemic allergic reaction that can be fatal, in many cases, if left untreated. Anaphylaxis can involve various areas of the body, such as the skin, respiratory tract, gastrointestinal tract, and cardiovascular system. Acute symptoms occur from within minutes to two hours after contact with the allergy-causing substance, but in rare instances onset may be delayed by as much as four hours. Contact with anaphylaxis-inducing agents, and the severity of the resulting anaphylactic reaction, can be extremely unpredictable. Accordingly, allergists recommend that persons who have a personal or family history of anaphylaxis be prepared to self-administer emergency treatment at all times. Additionally, adults charged with caring for children who are at risk for anaphylaxis should also be prepared to administer anti-anaphylactic first aid.

The symptoms of anaphylaxis include one or more of the following, generally within 1 to about 15 minutes of exposure to the antigen: agitation, a feeling of uneasiness, flushing, palpitations, paresthesias, pruritus, throbbing in the ears, coughing, sneezing, urticaria, angioedema, difficulty breathing due to laryngeal edema or brochospasm, nausea, vomiting, abdominal pain, diarrhea, shock, convulsions, incontinence, unresponsiveness and death. An anaphylactic reaction may include cardiovascular collapse, even in the absence of respiratory symptoms.

According to the Merck Manual, immediate treatment with epinephrine is imperative for the successful treatment of anaphylaxis (Merck Manual, 17.sup.th Ed., 1053-1054 (1999)). The recommended dose is about 0.01 mL/Kg in adults: usually about 0.3 to 0.5 mL of a 1:1000 dilution of epinephrine in a suitable carrier. While the dose may be given manually, such as either subcutaneously or intramuscularly, for example, in recent years automatic injectors have become an accepted first aid means of delivering epinephrine. It is recommended that persons at risk of anaphylaxis, and persons responsible for children at risk for anaphylaxis, maintain one or more automatic epinephrine injectors in a convenient place at all times. It is further recommended that, if the symptoms of anaphylaxis persist after the first dose of epinephrine is injected, the patient should be treated with a second dose of epinephrine (about 0.3 mL of the 1:1000 dilution).

Certain formulations of epinephrine are known. Epinephrine Injection, USP is a sterile, non-pyrogenic solution administered parenterally by the intravenous or intracardiac (left ventricular chamber) routes, or via endotracheal tube into the bronchial tree. Each milliliter (mL) of the 1:10,000 solution contains epinephrine 0.1 mg; sodium chloride 8.16 mg; sodium metabisulfite added 0.46 mg; citric acid, anhydrous 2 mg and sodium citrate, dihydrate 0.6 mg added as buffers. Sodium metabisulfite is used with Epinephrine formulations as a preservative. Sodium metabisulfite has been associated with severe allergic reactions. The formulation may contain additional citric acid and/or sodium citrate for pH adjustment. pH 3.3 (2.2 to 5.0).

Epinephrine Injection, USP is administered by intravenous injection and/or in cardiac arrest, by intracardiac injection into the left ventricular chamber or via endotracheal tube directly into the bronchial tree. The adult intravenous dose for hypersensitivity reactions or to relieve bronchospasm usually ranges from 0.1 to 0.25 mg (1 to 2.5 mL of 1:10,000 solution), injected slowly. Neonates may be given a dose of 0.01 mg per kg of body weight; for the infant 0.05 mg is an adequate initial dose and this may be repeated at 20 to 30 minute intervals in the management of asthma attacks.

In cardiac arrest, 0.5 to 1.0 mg (5 to 10 mL of 1:10,000 solution) may be given. During a resuscitation effort, 0.5 mg (5 mL) should be administered intravenously every five minutes. Intracardiac injection may be administered, if there has not been sufficient time to establish an intravenous route. The intracardiac dose usually ranges from 0.3 to 0.5 mg (3 to 5 mL of 1:10,000 solution). Also contemplated is dose delivery from vials with formulation comprising concentrated epinephrine solution and/or storage at room temperature.

Because of its catechol nucleus, epinephrine oxidizes easily and darkens slowly on exposure to air. Dilute solutions are partially stabilized by the addition of chlorobutanol and by reducing agents, such as sodium bisulfate or ascorbic acid. As the free amine, it is available in an oil solution for inhalation. Like other amines it forms salts with acids including the hydrochloride, the borate, and the bitartrate. The bitartrate has the advantage of being less acidic and is used in the eye because its solutions have a pH close to that of lacrimal fluid. Epinephrine is destroyed readily in alkaline solutions by aldehydes, weak oxidizing agents and oxygen of the air.

Along with its advantages, Epinephrine has several disadvantages that include a short duration of action, decomposition of its salts in solution, vasoconstriction action frequently followed by vasodilation and inactivity on oral administration.

The primary determinant of catecholamine stability in intravenous admixtures is the pH of the solution. Epinephrine hydrochloride is unstable in dextrose (5% in water) at a pH above 5.5. The pH of optimum stability is from about 3 to about 4. In one study, the decomposition rate increased two-fold (from 5 to 10% in 200 days at 30° C.) when the pH was increased form 2.5 to 4.5. Epinephrine hydrochloride is rapidly destroyed by alkali or by oxidizing agents including halogens, permanganates, chromate, nitrates, nitrites and salts of easily reducible metals such as iron, copper, and zinc. In alkaline solution and when exposed to air or light, it turns pink from oxidation to adenochrome and then brown from the formation of polymers. Epinephrine should not be mixed with aminophlline-containing solutions because of the alkalinity of these solutions. In one evaluation with aminophylline stored at 25° C., a color change was noted after about 8 hours and only 40% of the initial drug was still present in the admixture at 24 hours.

Instability has also been observed when drugs are combined with epinephrine. For example, when lidocaine hydrochloride is mixed with epinephrine hydrochloride the buffering capacity of the lidocaine raises the pH of the intravenous admixtures above 5.5, the maximum necessary for stability of the epinephrine, to about 6. Under these conditions, the epinephrine hydrochloride will begin to deteriorate within several hours.

### SUMMARY OF THE INVENTION

The present invention concerns particular formulations of epinephrine, which itself may also be referred to as epi, adrenaline, epinephrin, or adrenalin, for example, and it has a chemical formula of C₉H₁₃NO₃. The formulation is injectable, in particular embodiments. In certain aspects of the invention, the formulation has no sodium metabisulfite and has enhanced stability, such as being able to refrain from degradation before a certain period of time. In a particular embodiment, the formulation is enhanced to remain effective under any condition that would otherwise degrade the formulation, at least for an amount of time greater than that for an epinephrine formulation without a stability-enhancing agent, which in at least some cases may be an antioxidant. In specific cases, the formulation is enhanced to be stable in light, oxygen, and/or heat conditions and/or after an amount of time no less than 12 months. In specific cases, the formulation is enhanced to be stable for at least 12, 13, 14, 15, 16, 17, 18, 19, 20, 21, 22, 23, or 24 months, for example.

In particular aspects of the invention, the epinephrine formulation comprises ethylenediamine tetraacetic acid (also referred to as EDTA, H4EDTA, diaminoethanetetraacetic acid, edetic acid, edetate, edetate disodium, ethylenedinitrilotetraacetic acid, versene, or ethylene diamine tetracetic acid) and one or more antioxidants. Although any suitable antioxidant may be employed, in specific aspects the antioxidant is cysteine, citric acid, thioglycerol, ascorbic acid, acetylcysteine, or a combination thereof.

The formulations of the invention may be employed for any medical condition that epinephrine is useful. In particular embodiments, the epinephrine is utilized for anaphylaxis, cardiac arrest, or asthma, for example. Epinephrine is the preferred treatment for anaphylaxis even though the product contains sodium metabisulfite, which in other products may cause allergic-type reactions including anaphylactic symptoms or life-threatening asthma in certain susceptible persons.

The invention may be applied to any individual, but in specific embodiments the invention is useful for a mammal, including a human, dog, cat, horse, cow, goat, sheep, and so forth.

In a particular embodiment of the invention, there is an injectable pharmaceutical composition comprising epinephrine, EDTA and at least one antioxidant, wherein the antioxidant is selected from the group consisting of cysteine, citric acid, thioglycerol, acetylcysteine, and a combination thereof.

In another embodiment of the invention, there is an injectable pharmaceutical composition consisting essentially of epinephrine, EDTA, and at least one stability-enhancing agent, wherein the antioxidant is selected from the group consisting of cysteine, citric acid, thioglycerol, acetylcysteine, and a combination thereof.

In a further embodiment of the invention, there is a composition comprising a pharmaceutical composition comprising epinephrine, EDTA, and at least one antioxidant, wherein the antioxidant is selected from the group consisting of cysteine, citric acid, thioglycerol, acetylcysteine, and a combination thereof; and an injection apparatus. The pharmaceutical composition may be housed in the injection apparatus or housed separately from the injection apparatus. The injection apparatus may be further defined as a syringe. The injection apparatus may be further defined as an autoinjector.

In an additional embodiment of the invention, there is a method of improving at least one symptom of an epinephrine-requiring medical condition in an individual in need thereof, comprising injecting into the individual a formulation comprising epinephrine; EDTA; a pharmaceutically acceptable carrier; and at least one antioxidant, wherein the antioxidant is selected from the group consisting of cysteine, citric acid, thioglycerol, acetylcysteine, and a combination thereof. In a specific embodiment of the invention, the medical condition is anaphylaxis, asthma or cardiac arrest. The formulation may be housed in an injection apparatus or housed separately from an injection apparatus. The injection may be by an autoinjector. The injection may be in the thigh of the individual, intracardially into the individual, or endotracheally into the individual, for example.

In another embodiment of the invention, there is a method of treating anaphylaxis in an individual comprising injecting into the individual a formulation, said formulation comprising epinephrine; EDTA; a pharmaceutically acceptable carrier; and at least one antioxidant, wherein the antioxidant is selected from the group consisting of cysteine, citric acid, thioglycerol, acetylcysteine, and a combination thereof, wherein the formulation is injected by an auto-injector.

In a further embodiment of the invention, there is a method of treating a pediatric individual in need of treatment for anaphylaxis comprising injecting into the individual a formulation comprising epinephrine; EDTA; and at least one stability enhancing agent, wherein the stability enhancing agent is selected from the group consisting of cysteine, citric acid, thioglycerol, acetylcysteine, and a combination thereof.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 illustrates an epinephrine antioxidant study with cysteine formulations.

FIG. 2 illustrates an epinephrine antioxidant study with metabisulfite formulations.

### DETAILED DESCRIPTION OF THE INVENTION

### I. Definitions

In keeping with long-standing patent law convention, the words “a” and “an” when used in the present specification in concert with the word comprising, including the claims, denote “one or more.” Some embodiments of the invention may consist of, consist essentially of, have, and/or include one or more elements, method steps, and/or methods of the invention. It is contemplated that any method or composition described herein can be implemented with respect to any other method or composition described herein.

The term “anaphylaxis” as used herein refers to an acute hypersensitivity reaction as a result of exposure to an antigen, such as a previously encountered antigen or to a drug, for example. In specific embodiments, the symptoms may include rapidly progressing urticaria, respiratory distress, vascular collapse, systemic shock, and/or death.

The term “antioxidant” as used herein refers to a material that will prevent oxidation or be preferentially oxidized.

The term “autoinjector” as used herein refers to an apparatus wherein an individual may administer a formulation, such as a pharmaceutical formulation, to themselves. In specific embodiments, the autoinjector delivers a single dose. In other specific embodiments part or all of the autoinjector is disposable and/or portable. In specific embodiments, part or all of the autoinjector is opaque, and in further specific embodiments at least one part of the autoinjector that is opaque is the part that houses the pharmaceutical formulation. An auto-injector may be supplied separately from the pharmaceutical formulations, in alternative embodiments. The auto-injector, or any other injection apparatus, may comprise an exchangeable vessel for replacing the pharmaceutical formulation, such as an insert, cartridge, vial, and so forth. Such an exchangeable vessel may be glass or plastic, for example.

The term “epinephrine-requiring medical condition” as used herein refers to any medical condition wherein administration of epinephrine to an individual having the condition has a pharmacologically beneficial effect, such as improving at least one symptom of the medical condition. In specific aspects of the invention, the medical condition comprises acute hypersensitivity, such as anaphylactic reaction to one or more drugs; to animal serums, such as from a bee, wasp, or ant; to plant allergens, including peanuts; and to other allergens). In other embodiments, the medical condition comprises an asthmatic condition, such as to relieve bronchospasm. In additional embodiments, the medical condition comprises cardiac arrest or Stokes-Adams Syndrome.

The term “injectable” as used herein refers to a composition that is suitable to be delivered to an individual in an injection, such as with an injection device, including one that employs a syringe or a cartridge, which may be housed in a manual injection device or an auto-injection device, for example. In particular aspects, an injectable formulation is transferable by injection, as opposed to an aerosol, for example, which is not.

The term “pediatric individual” as used herein refers to an individual that is equal to or less than 18 years of age.

The term “stability-enhancing agent” as used herein refers to one or more agents that increase the stability of epinephrine, such as by increasing the amount of time before the epinephrine degrades to an unusable form. In specific embodiments, the agent prolongs the efficacy of epinephrine over time and/or upon subjection to conditions that degrade epinephrine to a form having reduced efficacy, such exemplary conditions being air, heat, and/or light. In particular embodiments, the stability-enhancing agent may be considered a preservative and/or antioxidant yet itself does not destabilize the formulation. Useful but exemplary antioxidants include one or more of cysteine, acetylcysteine, thioglycerol, citric acid, or a combination thereof. Therefore, in specific embodiments the agent is absent in a pharmaceutical formulation of the invention or is removable therefrom.

### II. Embodiments of the Invention

The present invention concerns compositions, devices for delivery of the compositions, routes of administration, and methods for treating any medical condition for which epinephrine is suitable for alleviating at least one symptom. Although the epinephrine formulations of the invention may be employed for any medical condition that would be improved thereby, in particular cases the medical condition for which the inventive composition is employed is an allergic reaction, including in the context of an allergic emergency, such as anaphylaxis, for example. Treatment of anaphylaxis concerns ameliorating or alleviating at least one symptom of anaphylaxis. In particular cases, the epinephrine formulation of the invention is employed to facilitate peripheral vascular resistance via alpha-stimulated vasoconstriction in cardiac dysrhythmias, such as cardiac arrest, that leads to impaired or totally inhibited cardiac output, such that blood is directed to the core of the body. Such a formulation and amount thereof is employed so long as there is no increased cardiac irritability to an medically unacceptable level.

Furthermore, the invention provides kits for treating epinephrine-required medical conditions, including allergic emergencies, such as anaphylaxis. The compositions of the present invention provide surprisingly-enhanced stability over other formulations. The stability enhancements provide benefits at least in terms of patient safety, enhanced shelf life, reduced waste, reduced cost, and/or improved convenience for the user. The compositions of the present invention provide formulations that are stable at room temperature and can be stored without the need for refrigeration. As such, the devices or kits can be placed on emergency crash carts and medical kits in clinics, emergency rooms, airplanes, schools, public places, restaurants, residences, on a person, or in urgent care centers or hospitals for easy access in emergency situations, for example.

Such treatment may be, and in most cases is, temporary, in particular embodiments of the invention. The formulations, methods, and kits of the invention are suitable for any setting in which epinephrine is required for medical purpose. In specific embodiments of the invention, the method or kit of the invention provides emergency relief from at least one symptom of anaphylaxis for a time sufficient for the patient to seek professional medical assistance. Thus, devices and kits of the invention are well-suited for inclusion in first aid kits in professional child care settings and homes, for example, especially where one or more persons at risk for anaphylaxis are known to dwell. They may also be conveniently carried by those who are at risk for anaphylaxis or those who are charged with caring for those who are at risk for anaphylaxis. They are also well-suited for inclusion in so-called crash carts in medical emergency rooms. The methods of the invention are suitable for treating persons who are at risk for allergic emergencies, such as anaphylaxis, in any of the exemplary aforementioned settings.

Epinephrine is typically administered in anaphylaxis by injection under the skin, or into a muscle, although any route of administration may be suitable. Injections can be given by a health care professional in a clinic or hospital setting. Alternatively, an auto-injector form, for example, provides a convenient applicator for the health-care professional or for self-administration by patients who suffer a severe allergic response to certain stimuli.

Epinephrine is commonly administered parenterally by means of an injection device. Common injection devices range from a simple manual syringe system to an auto-injector. A manual syringe system would include a syringe comprising a barrel and a plunger and an appropriately-sized needle. Such simple syringes may be adapted to accept pre-filled cartridges, be packaged with the drug formulation loaded in the syringe, or used with vials, for example. Formulated drugs such as epinephrine may be prepared and filled into ampoules, prefilled cartridges, syringes, or vials that may be single or multi-dose containers, for example.

An exemplary epinephrine formulation for use in the treatment of the medical condition may be delivered by intramuscular injection, in specific embodiments. In specific embodiments, the injection device would provide 2 mL of the epinephrine formulation of the invention and deliver a single dose of 0.3 mL epinephrine from a 1:1000 dilution (0.3 mg) of a sterile solution. Alternately, the injection device may provide 2 mL of the epinephrine formulation of the invention and deliver a single dose of 0.3 mL of epinephrine from a 1:2000 dilution (0.15 mg) of a sterile solution.

Automatic injectors, such as those exemplary devices disclosed in U.S. Pat. Nos. 5,358,489; 5,540,664; 5,665,071 and 5,695,472, for example, are known in the art. In general, all automatic injectors comprise a volume of epinephrine solution to be injected. In general, automatic injectors include a reservoir for holding the epinephrine solution, which is in fluid communication with a needle for delivering the drug, as well as a mechanism for automatically deploying the needle, inserting the needle into the patient, and delivering the dose into the patient. An illustrative and exemplary automatic injector is described in U.S. patent application Ser. No. 10/817,224 (U.S. Patent Application Publication No. 2005/0222539), which is incorporated herein in its entirety.

Exemplary injectors provide about 0.3 mL of epinephrine solution at about a concentration of either 0.5 or 1 mg of epinephrine per mL of solution (1:2000 or 1:1000, respectively). Each injector is capable of delivering a dose of epinephrine and any epinephrine left in the automatic injector (generally about 90% of the original volume of epinephrine) is unavailable for delivery and must be discarded.

Additionally, the automatic injectors deliver a uniform volume of 0.3 mL of epinephrine to the patient, whether that patient is an adult or a child. Whereas, the adult version delivers 0.3 mL of a 1:1000 dilution of epinephrine, the pediatric version delivers 0.3 mL of a 1:2000 dilution of epinephrine. This volume of medicine may present discomfort to smaller children, but any discomfort is offset by the life-saving nature of epinephrine in treating severe anaphylaxis. However, a further object of the invention is to fill the need for a composition and method of treating anaphylaxis in a person of less than about 30 Kg, wherein a smaller dose of epinephrine can be delivered to the patient.

Thus, treatment of a medical condition, such as an allergic emergency that includes treatment of anaphylaxis, for which the invention is especially well-suited. In addition, treatment of allergic emergency includes treatment of other allergic conditions that may be treated with epinephrine. For example, the symptoms of anaphylactoid reactions to drugs closely mimic those of anaphylaxis and are treated in a similar manner. In cases where it is not clear whether the reaction is a systemic immunological response (anaphylaxis) or a systemic toxic response (anaphylactoid reaction), the accepted first line of treatment is with epinephrine. In this sense, treatment of an allergic emergency encompasses treatment of anaphylaxis, an anaphylactoid response or both.

In some embodiments, the present invention provides a method of treating an allergic emergency, such as anaphylaxis, in a patient, comprising administering to the patient epinephrine. The method includes automatically injecting into a patient in need thereof a dose of epinephrine consisting essentially of about 0.3 mL of an epinephrine solution. The concentration of epinephrine in the epinephrine solution is about 1 mg of epinephrine per mL of solution. In some embodiments, in addition to the approximately 1 mg of epinephrine per mL, the solution also contains one or more inactive ingredients, such as EDTA, cysteine, acetylcysteine, thioglycerol, a pH buffer, an ingredient that provides isotonicity, or mixtures thereof.

The smaller dose of epinephrine 0.15 mg/unit dose (0.3 mL), is especially suitable for treating smaller patients with body weights less than 30 Kg. Thus, in some embodiments in which the dose is about 0.15 mg, the weight of the patient weighs less than about 30 Kg. In particular embodiments, the patient weighs less than about 15 Kg.

In certain aspects, the epinephrine formulation of the present invention prepared for administration as a sterile aqueous solution combines EDTA in the exemplary range from 0.01% to 0.048%, cysteine in the exemplary range from 0.05% to 0.10%, with at least one of the optional components including, for example, citric acid in the exemplary range from 0.02% to 1.3%, thioglycerol in the exemplary range from 0.10% to 1.0%, or acetylcysteine in the exemplary range form 0.10% to 1.0%.

In certain aspects of the invention, there is an epinephrine composition comprising a chelating agent (EDTA) in combination with at least one antioxidant. The present invention shows that in particular embodiments EDTA alone is less effective than combinations with antioxidants. In a specific aspect, the combination of cysteine and citric acid with epinephrine is less effective than the combination of cysteine or cysteine/citric acid with epinephrine and EDTA, for example. In particular cases, the epinephrine combinations of the invention exhibit a synergistic effect.

In at least some embodiments of the invention, the epinephrine formulations employ an improved purity over known formulations, such as, for example, by comprising fewer epinephrine degradation products in the formulation as a result of the combination of EDTA and antioxidants.

In certain cases, certain ranges for the components of the formulation of the invention are employed. In specific embodiments, a range of EDTA that may be employed is 0.01 to 0.2%, and in particular embodiments it is 0.01 to 0.05%. In additional specific embodiments, a range of cysteine that may be employed is 0.05 to 2.6%, and in particular embodiments it is 0.1 to 0.5%. In further specific embodiments, a range of citric acid that may be employed is 0.05 to 1.3%, although in particular embodiments it is 0.01 to 0.5%. In additional specific embodiments, a range of thioglycerol that may be employed is 0.05 to 1.0, although in particular embodiments it is 0.1 to 0.5%. In other specific embodiments, a range of ascorbic acid that may be employed is 0.05 to 2.0%, although in particular embodiments a range of 0.2 to 0.5% may be utilized. In further specific embodiments, a range of acetylcysteine that may be utilized is 0.05 to 1.0%, although in particular embodiments the range is 0.1 to 0.5%.

### III. Pharmaceutical Preparations

Pharmaceutical compositions of the present invention comprise an effective amount of one or more epinephrine formulations. The formulation may be dissolved or dispersed in a pharmaceutically acceptable carrier. The carrier may or may not be the stability-enhancing agent of the invention. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one epinephrine formulation and/or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The epinephrine formulation may be administered in liquid form, and whether it need to be sterile for such routes of administration as injection. The present invention can be administered in any suitable manner although in specific embodiments its administration is intravenously, intradermally, intrathecally, intraarterially, intraperitoneally, intramuscularly, subcutaneously, locally, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

Upon formulation, solutions will be administered in a manner compatible with the dosage formulation and in such amount as is therapeutically effective. The formulations are easily administered in a variety of dosage forms such as formulated for parenteral administrations, such as injectable solutions. Further in accordance with the present invention, the composition of the present invention suitable for administration is provided in a pharmaceutically acceptable carrier with or without an inert diluent. The carrier should be assimilable and includes a liquid carrier. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In further embodiments, epinephrine formulations may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### IV. Kits of the Invention

Any of the compositions described herein may be comprised in a kit. In a non-limiting example, an epinephrine formulation of the invention may be comprised in a kit. The kits will thus comprise, in suitable container means, an epinephrine formulation of the present invention.

The kits may comprise a suitably aliquoted epinephrine formulation. In certain cases, the formulation comprises EDTA and one or more of acetylcysteine, cysteine, thioglycerol, or citric acid. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the epinephrine formulation and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. The epinephrine formulation may also be formulated into a syringeable composition. In which case, the container means may itself be a syringe, pipette, and/or other such like apparatus, from which the formulation may be applied to an appropriate area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the invention may also comprise, and/or be packaged with, an instrument for assisting with the injection/administration and/or placement of the ultimate epinephrine formulation into the body of an animal. Such an instrument may be a syringe, auto-injector, or any such medically approved injection delivery vehicle.

### V. Examples

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of Exemplary Formulation

Prepare epinephrine for injection 1:1000 by adding epinephrine of 1.0 mg, 0.1mg of EDTA, 1.0 mg of cysteine to 0.8 ml of water for injection, adjusting the pH to 3.5±0.2 with dilute HCL or NaOH and adding sodium chloride to adjust the osmolarity to between 275 and 300 mOsm/L and QS to 1 ml volume with water for injection. The final solution is filtered through a 0.2 micron sterile filter into individual sterile vials or single-use sterile syringes.

### Example 2

### Preparation of Exemplary Formulation Comprising Citric Acid and Cysteine

Prepare epinephrine for injection 1:1000 by adding epinephrine of 1.0 mg, 0.1mg of EDTA, 1.0 mg of cysteine and 0.2 mg citric acid to 0.8 ml of water for injection, adjusting the pH to 3.5±0.2 with dilute HCL or NaOH and adding sodium chloride to adjust the osmolarity to between 275 and 300 mOsm/L and QS to 1 ml volume with water for injection. The final solution is filtered through a 0.2 micron sterile filter into individual sterile vials or single-use sterile syringes.

### Example 3

### Preparation of Exemplary Formulation Comprising Cysteine and Thioglycerol

Prepare epinephrine for injection 1:1000 by adding epinephrine of 1.0 mg, 0.1mg of EDTA, 1.0 mg of cysteine, and 1.0 mg thioglycerol to 0.8 ml of water for injection adjusting the pH to 3.5±0.2 with dilute HCL or NaOH and adding sodium chloride to adjust the osmolarity to between 275 and 300 mOsm/L and QS to 1 ml volume with water for injection. The final solution is filtered through a 0.2 micron sterile filter into individual sterile vials or single-use sterile syringes.

### Example 4

### Preparation of Exemplary Formulation Comprising Cysteine and Acetylcysteine

Prepare epinephrine for injection 1:1000 by adding epinephrine of 1.0 mg, 0.1mg of EDTA, 1.0 mg of cysteine and 1.0 mg acetylcysteine to 0.8 ml of water for injection, adjusting the pH to 3.5±0.2 with dilute HCL or NaOH and adding sodium chloride to adjust the osmolarity to between 275 and 300 mOsm/L and QS to 1 ml volume with water for injection. The final solution is filtered through a 0.2 micron sterile filter into individual sterile vials or single-use sterile syringes.

### EXAMPLE 5

### Present Exemplary Inventive Formulations Vs. Related Art Formulations

There are several marketed epinepharine IM injection formulations. Most use a bisulfite antioxidant. Examples below present two such formulations.

| Ingredient | Amount |
|---|---|
| EpiPen ® Formulation: | |
| Epinephrine | 0.30 mg |
| Sodium Metabisulfite | 0.50 mg |
| Sodium Chloride | 1.80 mg |
| Hydrochloric Acid | Qs to pH 2.2 to 5.0 |
| Water for Injection | Qs to 0.3 ml |
| TwinJect ™ Formulation: | |
| Epinephrine | 0.30 mg |
| Sodium bisulfite | 0.45 mg |
| Sodium Chloride | 2.60 mg |
| Chlorobutanol | 1.50 mg |
| Water for Injection | Qs to 0.3 ml |

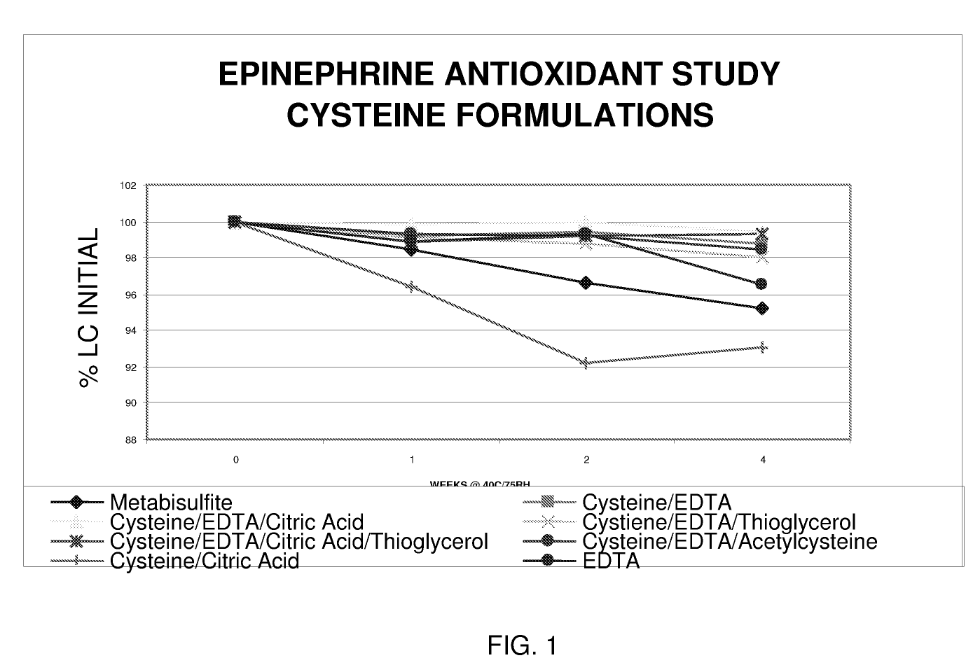

Comparative stability data is as follows: formulations comprising cysteine were significantly better than Metabisulfite formulation used in EpiPen®. Studies were conducted to evaluate the effect of removing Metabisulfite while adding cysteine and other additives at accelerated stability conditions of 40° C./75% RH. Stability data compared to metabisulfite formulation are given in FIG. 1, wherein percentage of the initial labeled concentration normalized to 100% is provided on the y-axis, and the corresponding data in tabular form is provided in Table 1.

TABLE 1
Epinephrine Antioxidant Study with Cysteine Formulations

| time | | | | | | | | |
|---|---|---|---|---|---|---|---|---|
| weeks | metabisulfite | Cys/EDTA | CYS/citric/EDTA | cys/thio/EDTA | cys/thio/citric/EDTA | CYS/EDTA/Acty | Cys/citric | EDTA |
| 0 | 100 | 100 | 100 | 100 | 100 | 100 | 100 | 100 |
| 1 | 98.4 | 99.2 | 99.8 | 99.1 | 98.9 | 99.3 | 96.4 | 98.9 |
| 2 | 96.6 | 99.4 | 100 | 98.8 | 99 | 99 | 92.2 | 99.3 |
| 4 | 95.2 | 98.8 | 99.4 | 98 | 99 | 98 | 93.1 | 96.5 |

The cysteine formulation containing just EDTA, or EDTA and citric acid, or EDTA and thioglycerol, or EDTA and acetylcysteine, or EDTA, citric acid and thioglycerol all were significantly better than metabisulfite alone.

An object of the invention is the use of the epinephrine formulation of the invention for injection under the skin, into a muscle or into a vein. Injections can be given by a health care professional in a clinic or hospital setting. Alternatively the formulation of the invention may be used with an auto-injector to provide a convenient applicator for the health-care professional or for self-administration by patients who suffer a severe allergic response to certain stimuli.

To use the auto-injector for anaphylaxis; one typically will remove a safety cap, place the tip of the injector on the thigh at a right angle to your leg, press the tip hard into your leg and activate the injection function. Hold the applicator in place for several seconds and then remove and safely throw away. Massage the leg for 10 seconds.

Additionally, the automatic injectors deliver a uniform volume of 0.3 mL of epinephrine to the patient, whether that patient is an adult or a child. The pediatric version delivers 0.3 mL of a 1:2000 dilution of epinephrine. This volume of medicine can present discomfort to smaller children, but any discomfort is offset by the life saving nature of epinephrine in treating severe anaphylaxis. However, a further object of the invention is to fill the need for a composition and method of treating anaphylaxis in a person of less than about 15 Kg, wherein a smaller volume of epinephrine can be delivered to the patient.

An object of the invention is the epinephrine formulation of the invention prepared as a solution (1:1000 or 1:2000 dilutions) for SC or IM applications. The normal doses would include the following exemplary formulations:

Children 1 month to 12 years of age:

0.01 mg/kg (0.01 ml/kg) or 0.004 mg/lb (0.004 ml/lb) not to exceed 0.3-0.5 mg (0.3-0.5 ml) in a single dose. The dosing may be repeated as necessary but should not exceed three doses. Each dose from a typical auto-injector applicator would contain for example 0.15 mg epinephrine.

Adults and children over 12 years of age:

0.3-0.5 mg (0.3-0.5 ml) every 20 minutes to 4 hours as necessary not to exceed 1 mg (1 ml) in a single dose. Each dose from atypical auto-injector applicator would contain for example 0.3 mg epinephrine.

### Example 6

### Epinephrine Antioxidant Study

The present example concerns a study comparing epinephrine with or without particular metabisulfite formulations. Table 2 below shows the stability of the particular formulations for up to four weeks.

TABLE 2
Epinephrine Antioxidant Study: Metabisulfite Formulations

| time | | | | METABI/ | |
|---|---|---|---|---|---|
| weeks | metabisulfite | EDTA | Metabi/EDTA | ascorbic | ascorbic |
| 0 | 100 | 100 | 100 | 100 | 100 |
| 1 | 98.4 | 98.9 | 99.7 | 100 | 97.1 |
| 2 | 96.6 | 99.3 | 99.2 | 99.6 | 97 |
| 4 | 95.2 | 96.5 | 99 | 99.8 | 93 |

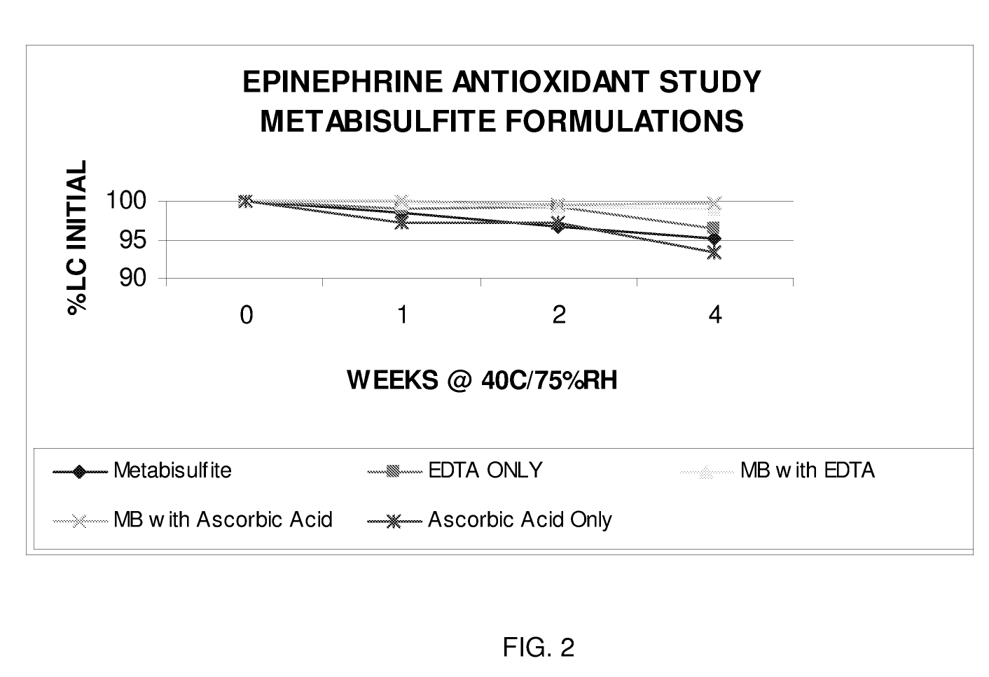

FIG. 2 illustrates the data provided in Table 2.

### REFERENCES

All patents and publications mentioned in the specification are indicative of the levels of those skilled in the art to which the invention pertains. All patents and publications are herein incorporated by reference in their entirety to the same extent as if each individual publication was specifically and individually indicated to be incorporated by reference.

### Patents and Patent Applications

U.S. Pat. No. 5,358,489
U.S. Pat. No. 5,540,664
U.S. Pat. No. 5,665,071
U.S. Pat. No. 5,695,472
U.S. Pat. No. 5,466,468
U.S. Pat. No. 5,756,353
U.S. Pat. No. 5,804,212
U.S. Pat. No. 5,725,871
U.S. Pat. No. 5,780,045
U.S. Pat. Appl. Publ. 20050222539

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

We claim:

1. An injectable pharmaceutical composition comprising epinephrine, EDTA, and at least one antioxidant, wherein the antioxidant is selected from the group consisting of cysteine, citric acid, thioglycerol, acetylcysteine, and a combination thereof.

2. The composition of claim 1, wherein the antioxidant is cysteine.

3. The composition of claim 1, wherein the antioxidant is citric acid.

4. The composition of claim 1, wherein the antioxidant is thioglycerol.

5. The composition of claim 1, wherein the antioxidant is acetylcysteine.

6. A kit, comprising:
a pharmaceutical composition comprising epinephrine, EDTA, and at least one antioxidant, wherein the antioxidant is selected from the group consisting of cysteine, citric acid, thioglycerol, acetylcysteine, and a combination thereof; and
an injection apparatus.

7. The kit of claim 6, wherein said pharmaceutical composition is housed in the injection apparatus or is housed separate from the injection apparatus.

8. The kit of claim 6, wherein said injection apparatus is further defined as being selected from the group consisting of a syringe and an autoinjector.

9. The kit of claim 6, wherein the antioxidant is cysteine.

10. The kit of claim 6, wherein the antioxidant is citric acid.

11. The kit of claim 6, wherein the antioxidant is thioglycerol.

12. The kit of claim 6, wherein the antioxidant is acetylcysteine.

13. A method of improving at least one symptom of an epinephrine-requiring medical condition in an individual in need thereof, comprising injecting into the individual a formulation comprising:
epinephrine;
EDTA;
a pharmaceutically acceptable carrier; and
at least one antioxidant, wherein the at least one antioxidant is selected from the group consisting of cysteine, citric acid, thioglycerol, acetylcysteine, and a combination thereof.

14. The method of claim 13, wherein said formulation is housed in an injection apparatus or is housed separately from an injection apparatus.

15. The method of claim 13, wherein said injecting is by an autoinjector.

16. The method of claim 13, wherein the antioxidant is cysteine.

17. The method of claim 13, wherein the antioxidant is citric acid.

18. The method of claim 13, wherein the antioxidant is thioglycerol.

19. The method of claim 13, wherein the antioxidant is acetylcysteine.

20. The method of claim 13, wherein the injection is in the thigh of the individual, is intracardially into the individual, or is endotracheally into the individual.

21. A method of treating anaphylaxis in an individual comprising injecting into the individual a formulation, said formulation comprising:
epinephrine;
EDTA;
a pharmaceutically acceptable carrier; and
at least one antioxidant, wherein the at least one antioxidant is selected from the group consisting of cysteine, citric acid, thioglycerol, acetylcysteine, and a combination thereof, wherein the formulation is injected by an auto-injector.

22. A method of treating a pediatric individual in need of treatment for anaphylaxis comprising injecting into the individual a formulation comprising:
epinephrine;
EDTA; and
at least one antioxidant, wherein the at least one antioxidant is selected from the group consisting of cysteine, citric acid, thioglycerol, acetylcysteine, and a combination thereof.
